(19) United States
(12) Patent Application Publication
(10) Pub. No.: US 2006/0030719 A1
(43) Pub. Date: Feb. 9, 2006

(54) Cis-3,5-disubstituted-dihydro-furan-2-ones and the preparation and use thereof

(76) Inventors: Paul Joseph Fagan, Wilmington, DE (US); Charles J. Brandenburg, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/910,495

(22) Filed: Aug. 3, 2004

(51) Int. Cl. C07D 307/20

(52) U.S. Cl. 549/263

(57) ABSTRACT

The present invention relates to an improved process to prepare cis-3-dihydrocarbylmethano-5-hydrocarbyidihydro-furan-2-ones. The present invention also relates to novel compositions of matter comprising enantiomerically pure cis-3-dihydrocarbylmethano-5-hydrocarbyldihydro-furan-2-ones, being the (3S,5S), (3R,5R), (3S,5R), or (3R,5S) optically pure isomers, and a new, more cost efficient process to prepare said optically pure isomers.

### FIELD OF THE INVENTION

The present invention relates to the novel preparation of pure cis-3,5-disubstituted-dihydro-furan-2-ones and also relates to novel compositions of matter, those being the chemically pure and enantiomerically pure cis-isomers of 3,5-disubstituted-dihydro-furan-2-ones and the use of said compositions as flavors or fragrances.

### TECHNICAL BACKGROUND

There is much interest in the preparation of substituted dihydro-furanones because of their utility in fragrance and flavoring applications. There is a large body of prior art that describes the synthesis of 3,5-disubstituted-dihydro-furan-2-ones. Examples of prior art describing processes to prepare 3,5-disubstituted-dihydro-furan-2-ones include acid catalyzed hydrolysis of alkenyl carboxylic acids and esters (Braun, Chem. Ber. Vol. 70, pp 1252 (1937)), alkylation of a lactone enolate salt (Jelinski, Z., Kowalczuk, M., Kurcok, P., Grzegorzek, M., Ermel, J. J. Org. Chem. Vol. 52, pp 4601-4602 (1987)), alkylation of epoxides by acid or ester enolate salts (Hirai, Y. Yakota, K., Yamazaki, T., Momose, T. Heterocycles, Vol. 30, pp 1101-1119 (1990)), hydrolysis of alkene nitriles (Tiecco, M., Testaferri, L., Bartoli, D., Synth. Commun. Vol. 19, pp 2817-2824, (1989)), oxidative carbonylation of alkenols (Alper, H., Leonard, D., J. Chem. Soc., Chem. Comm. pp 511-512 (1985)), and carbonylation of alkynes in the presence of methyl iodide (Wang, J.-X., Alper, H. J. Org. Chem. Vol. 51, pp 273-275 (1986). These preparations of 3,5-disubstituted-dihydro-furan-2-ones all produce mixtures of cis and trans stereoisomers. A preparation of cis-3,5-disubstituted-dihydro-furan-2-ones has been accomplished by Rebrovic and Harris (U.S. Pat. Nos. 4,980,342; 5,231,192) by reacting-the anion of ethylacetoacetate with epoxides to form 3-acyl-5-alkyl-dihydro-furan-2-one derivatives in 48% yield; the 3-acyl-5-alkyl-dihydro-furan-2-ones were then treated with base in the presence of an aldehyde in a refluxing solvent to azeotrope the water from the reaction forming the 3-alkylidene-5-alkyl-dihydro-furan-2-ones in typically 50% yield after isolation. Thus the overall yield of 3-alkylidene-5-alkyl-dihydro-furan-2-one from these two combined steps was typically in the range of 20-32% yield. For the economy of a process, it would be advantageous to have an overall larger molar yield from the starting materials. It would also be advantageous not to require azeotropic removal of water during the process, nor require strong alkali base and the aldehyde reactant to contact each other which may degrade the aldehyde and thus lower the yield. All of these advantages are realized by the present invention.

It is well known in the art that enantiomerically pure stereochemical isomers will have different flavor and fragrance characteristics than the racemic mixture of both isomers, or when compared with each other (for example, U.S. Pat. No. 6,495,729 B2). Therefore, it is advantageous to have a method to prepare cis-3-(dihydrocarbylmethano)-5-(hydrocarbyl)-dihydro-furan-2-ones with high enantiomeric excess of the (3S,5S), (3R,5R), (3S,5R), or (3R,5S) stereoisomeric configuration. Enantiomerically pure cis-(3R,5S)-3,5-dimethyl-dihydro-furan-2-one has been prepared by reaction of (S)-propylene oxide with the anion of the diester of malonic acid producing (5S)-methyl-butyrolactone, which was followed by formylation of the 3-position, reduction to the (S5)-3-(hydroxymethyl)- 5-methyl-dihydro-furan-2-one, dehydration to the (S5)-3-methylene-5-methyl-dihydro-furan-2-one, and hydrogenation over 10% palladium on carbon to yield pure cis-(3R,5S)-3,5-dimethyl-dihydro-furan-2-one (White, J. D., Amedio, Jr., J. C., J. Org. Chem., Vol. 54, pp 738-743 (1989)). This process to make pure cis-(3R,5S)-3,5-dimethyl-dihydro-furan-2-one uses expensive reagents. Other processes to prepare enantiomerically pure cis-3,5-dimethyl-dihydro-furan-2-one compositions that require multiple steps and expensive reagents have also been reported (Kang, S. K.; Lee, D. H. Synlett, pp 175-176 (1991); Hirai, Y.; Yokota, K. Sakai, H.; Yamazaki, T.; Momose, T. Heterocycles, Vol. 29 pp1865-1869 (1989); Tiecco, M.; Tingoli, M; Testaferri, L.; Bartoli, D. Synthetic Communications, Vol 19, pp 2817-2824 (1989)). The composition cis-(3S,5S)-3-methyl-5-phenyl-dihydro-furan-2-one has been prepared in enantiomerically pure form from an expensive organometallic iron reagent (Davies, S. G.; Polywka, R.; Warner, P. Tetrahedron, Vol. 46, pp 4847-4856 (1990)). These former compositions are the only enantiomerically pure cis-3-(dihydrocarbylmethano)-5-(hydrocarbylydihydro-furan-2-ones described in the art. The present invention provides for new enantiomerically pure compositions cis-(3S,5S)-3-(dihydrocarbylmethano)-5-(hydrocarbyl)-dihydro-furan-2-ones, cis-(3R,5R)-3-(dihydrocarbylmethano)-5-(hydrocarbyl)-dihydro-furan-2-ones, cis-(3S,5R)-3-(dihydrocarbylmethano)-5-(hydrocarbyl)-dihydro-furan-2-ones, and cis-(3R, 5S)-3-(dihydrocarbylmethano)-5-(hydrocarbyl)-dihydro-furan-2-ones. A novel more economical process to prepare enantiomerically pure cis-3-(dihydrocarbylmethano)-5-(hydrocarbyl)-dihydro-furan-2-ones is realized in the present invention as described in detail to follow.

### BRIEF SUMMARY OF THE INVENTION

The present invention relates to a novel process to prepare compounds represented by formula I; 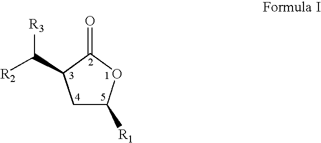
wherein R₁, and the group at position 3 of the lactone ring (containing R₂ and R₃) have a cis orientation with respect to each other; R₁ is selected from linear, branched, cyclic, bicyclic, saturated and unsaturated hydrocarbyl or substituted hydrocarbyl radicals; R₂ and R₃ are independently selected from hydrogen or linear, branched, cyclic, bicyclic, saturated and unsaturated hydrocarbyl or substituted hydrocarbyl radicals. The novel process is described by the sequence of steps in Scheme 1, and comprises the steps of: (a) contacting a lactone of formula 11 with an oxalic acid diester in the presence of a base and a solvent to form an intermediate mixture comprising a compound of formula III and isolating the compound of formula III from the intermediate mixture; (b) treating the isolated compound of formula III with an aldehyde or a ketone and isolating a compound of formula IV from the product mixture; and (c) hydrogenating the compound of formula IV in the presence of a catalyst and optionally a solvent and isolating a pure compound of formula I; wherein, R is a hydrocarbyl or substituted hydrocarbyl group, X⁺ is a cation, and wherein the pure compound is defined as greater than 95 percent pure by gas chromatographic analysis. The product of the process, formula I, is a racemic mixture of the optical isomers.

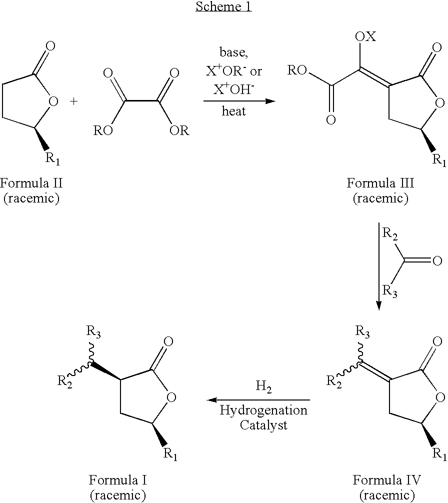

Further, the present invention relates to novel compositions of matter represented by formula 1 wherein R₁, and the group at position 3 of the lactone ring (containing R₂ and R₃) have a cis orientation with respect to each other; R₁ comprises a group selected from the groups consisting of linear, branched, cyclic, bicyclic, saturated and unsaturated hydrocarbyl groups; R₂, and R₃ are independently selected from the groups consisting of hydrogen and linear, branched, cyclic, bicyclic, saturated and unsaturated hydrocarbyl groups excluding compounds wherein R₂ and R₃ are both H, when R₁ is methyl or phenyl; and said composition contains a molar ratio of cis:trans stereoisomers greater than 49:1; and said compositions have greater than 95 percent enantiomeric purity, being the (3S,5S), (3R,5R), (3S,5R), or (3R,5S) optically pure isomers.

Further, the novel compositions of the present invention may be prepared to greater than 95 percent purity by gas chromatographic (GC) analysis.

Further, the present invention relates to an additional process to produce the optically pure, (3S,5S), (3R,5R), (3S,5R), or (3R,5S), cis isomer of compounds of formula I, comprising the same steps as the previously described process, with the exception that the starting compound Formula II is either the pure (R) or (S) stereoisomer.

Further the present invention relates to methods to improve, enhance, or modify the flavor or fragrance of a product formulation; methods to improve or modify the rheology of an oil, hydrocarbon, petroleum product; methods of formulating a cosmetic product; and methods of formulating a liquid detergent or cleaning product.

### BRIEF DESCRIPTION OF THE DRAWING(S)

FIG. 1: Structure of cis-3-octyl-5-methyl-dihydro-furan-2-one as determined by X-ray crystallography analysis.

### DETAILED DESCRIPTION OF THE INVENTION

The present inventors have discovered a novel process for preparing a compound represented by formula I: 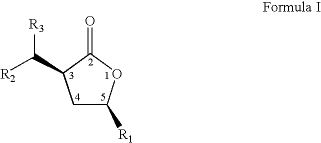
wherein R₁, and the group at position 3 of the lactone ring (containing R₂ and R₃) have a cis orientation with respect to each other; R₁ comprises a group selected from the groups consisting of linear, branched, cyclic, bicyclic, saturated and unsaturated hydrocarbyl groups; R₂, and R₃ are independently selected from the groups consisting of hydrogen and linear, branched, cyclic, bicyclic, saturated and unsaturated hydrocarbyl groups; and said composition contains a molar ratio of cis:trans stereoisomers greater than 49:1. The novel process is described by the sequence of steps in Scheme 1, and comprises the steps of:

(a) contacting a lactone of formula II with an oxalic acid diester in the presence of a base and a solvent to form an intermediate mixture comprising a compound of formula III and isolating the compound of formula III from the intermediate mixture;

(b) treating the isolated compound of formula III with an aldehyde or ketone, to form a second intermediate mixture comprising a compound of formula IV and isolating the compound of formula IV from the second intermediate mixture; and

(c) hydrogenating the compound of formula IV in the presence of a catalyst and optionally a solvent to form a product mixture comprising a compound of formula I and isolating a pure compound of formula I from the product mixture; 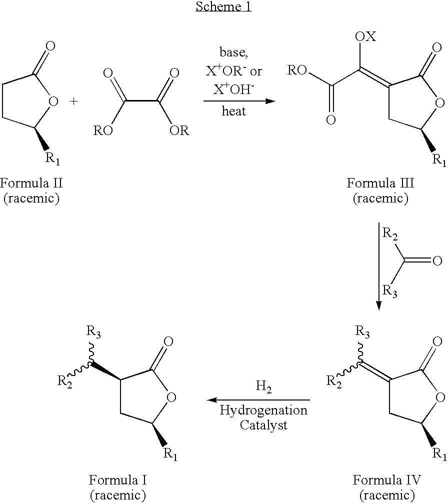
wherein, R is a hydrocarbyl or substituted hydrocarbyl group, and X⁺ is a cation. The product of the process, formula I, is a racemic mixture of the optical isomers. By pure compound of formula I is meant that the purity of the isolated compound is at least about 95 percent as determined by gas chromatographic analysis.

The present inventors have also discovered new compositions of matter comprising compounds represented by formula 1 wherein, R₁ and the group at position 3 of the lactone ring (containing R₂ and R₃) have a cis orientation with respect to each other; R₁ is selected from linear, branched, cyclic, bicyclic, saturated and unsaturated hydrocarbyl or substituted hydrocarbyl radicals; R₂ and R₃ are independently selected from hydrogen or linear, branched, cyclic, bicyclic, saturated and unsaturated hydrocarbyl radicals excluding compounds wherein R₂ and R₃ are both H, when R₁ is methyl or phenyl; said composition contains a molar ratio of the cis to trans stereoisomers of greater than 49:1; and said composition is greater than 95 percent enantiomerically pure, being the (3S,5S), (3R,5R), (3S,5R), or (3R,5S) optically pure isomer.

A “hydrocarbyl group” is a univalent group containing only carbon and hydrogen. If not otherwise stated, it is preferred that hydrocarbyl groups herein contain 1 to about 30 carbon atoms.

By “substituted hydrocarbyl” herein is meant a hydrocarbyl group, which contains one or more substituent groups, which are inert under the process conditions to which the compound containing these groups is subjected. The substituent groups also do not substantially interfere with the process. If not otherwise stated, it is preferred that substituted hydrocarbyl groups herein contain 1 to about 30 carbon atoms. Included in the meaning of “substituted” are heteroaromatic rings.

Additionally, the composition of matter comprising the compounds represented by formula I may be prepared to possess an overall purity of greater than 95 percent as determined by gas chromatographic analysis. The percent purity is calculated from the chromatogram as an area percent of the main component peak relative to the summed area for all peaks in the chromatogram.

Additionally, the inventors have discovered a novel process to prepare the composition of formula I wherein, R₁, and the group at position 3 of the lactone ring (containing R₂ and R₃) have a cis orientation with respect to each other; R₁ is selected from linear, branched, cyclic, bicyclic, saturated and unsaturated hydrocarbyl radicals; R₂ and R₃ are independently selected from hydrogen or linear, branched, cyclic, bicyclic, saturated and unsaturated hydrocarbyl radicals excluding compounds wherein R₂ and R₃ are both H, when R₁ is methyl or phenyl; and in addition, wherein said composition of formula I comprises greater than 96 percent optically purity, being the (3S,5S), (3R,5R), (3S,5R), or (3R,5S) optically pure isomers. The process is described by the sequence of steps in Scheme 2, wherein the starting gamma-methyl-gamma-butyrolactone (Formula II) is greater than 96% enantiomeric excess the (R) or (S) stereoisomer. The novel process yields a single stereoisomer with the cis orientation of R₁ and the group at position 3 of the lactone ring (containing R₂ and R₃) rather than a mixture of two possible stereoisomers, and further affords a route to the optically pure isomer, being the (3S,5S), (3R,5R), (3S,5R), or (3R,5S) optically pure isomers that are not known.

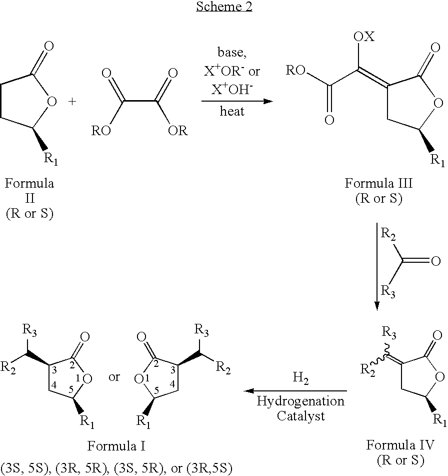

The process for preparing the enantiomerically pure composition of formula I comprises the steps: (a) contacting an optically pure stereoisomer of a lactone of formula II with an oxalic acid diester in the presence of a base and a solvent to form an intermediate mixture comprising an optically pure compound of formula III and isolating the optically pure compound of formula III from the intermediate mixture; (b) treating the isolated optically pure compound of formula III with an aldehyde or ketone, to form a second intermediate mixture and isolating an optically pure compound of formula IV from the second intermediate mixture; and (c) hydrogenating the optically pure compound of formula IV in the presence of a catalyst and optionally a solvent to form a product mixture and isolating an enantiomerically pure compound of formula I from the product mixture.

Lactones of formula II from Scheme 1 (racemic mixture) are commercially available from Aldrich, St. Louis, Mo. Lactones of formula II from Scheme 2, which are the pure (R) or (S) isomer may be prepared from a malonic acid diester by reaction with a base at elevated temperature to form the malonic acid diester enolate salt; and then reacting the salt with either an (R) or (S) epoxide to form the lactone. The details of the preparation are given in Hedenstroem, Erik; Hoegberg, Hans-Erik; Wassgren, Ann-Britt; Bergstroem, Gunnar; Loefqvist, Jan; Tetrahedron; 48; 1992; pp. 3139-3146.

The two processes of the present invention may be run under the same conditions, the detailed conditions are provided below, while the starting compound is different; one starting compound being a racemic mixture and the other starting compound being a pure (R) or (S) stereoisomer.

The first step of the processes is conducted at a temperature of at least about 25° C. and a pressure less than or equal to 2000 psi, preferably about 75° C. and about atmospheric pressure. The reaction may optionally run at higher temperatures, at about 100° C. to about 120° C. under higher pressures of about 700 psi. The reaction may optionally employ an organic solvent and use a phase transfer catalyst. The first step of the process can employ any number of solvents or combinations thereof; these include but are not limited to methanol, ethanol and isopropanol.

The R group of the oxalic acid diester of the first step in the processes may be a hydrocarbyl or substituted hydrocarbyl group, preferably, a methyl or ethyl group.

The base of the first step of the processes may be selected from the group consisting of metal alkoxides, metal oxides, hydroxides, carbonates and phosphates. The metal alkoxides, oxides, hydroxides, carbonates and phosphates employed herein may be used as solutions, powders, granules, or other particulate forms, or may be supported on an essentially inert support as is common in the art of catalysis. Representative bases include but are not limited to sodium methoxide, sodium ethoxide, sodium isopropoxide, sodium n-butoxide, potassium carbonate, cesium carbonate, sodium carbonate, barium carbonate, sodium hydrogen carbonate, magnesium oxide, barium oxide, barium hydroxide, lanthanum oxide, potassium hydroxide, cadmium oxide, rubidium oxide, lithium hydroxide, strontium hydroxide, sodium hydroxide, calcium hydroxide, potassium hydroxide, potassium phosphate and mixtures thereof.

The second step of the processes of the present invention is conducted at a temperature of at least about 0° C. and a pressure less than or equal to 2000 psi, preferably about 10° C. and about atmospheric pressure. The second step of the processes can employ any number of solvents or combinations thereof, these include but are not limited to water, toluene, xylenes, hexanes, ethyl acetate, chlorobenzene, 1,2-dichlorobenzene, acetonitrile, methylene chloride, acetone, methyl ethyl ketone, dimethylacetamide, chloroform, chlorobutane, and benzene.

The aldehyde or ketone of the second step of the novel processes may be represented by the formula R₂COR₃, wherein R₂ and R₃ are independently selected from hydrogen and linear, branched, cyclic, bicyclic, saturated and unsaturated hydrocarbyl or substituted hydrocarbyl radicals. The hydrocarbyl or substituted hydrocarbyl groups of R₂ and/or R₃ may contain from one to 30 carbon atoms.

The first and second steps of the inventive processes have been disclosed previously with respect to a process to produce alpha-methylenelactones and alpha-substituted hydrocarbylidene lactones in U.S. Pat. No. 6,531,616, incorporated herein by reference.

The third step of the processes, hydrogenation, may be conducted at a temperature of at least about 20° C. up to about 200° C. and a pressure less than or equal to 2000 psi, preferably about atmospheric pressure. The contact time for the hydrogenation step may be from about 15 minutes to about 12 hours.

The hydrogenation catalyst of the third step of the processes can include one or more metals selected from Group 8 elements from the Periodic Table of Elements, more preferably, the group consisting of iridium, nickel, palladium, platinum, rhenium, rhodium and ruthenium. The metal catalyst can optionally be supported on a catalyst support. The metal can be deposited on the support using any method known in the art. Preferably, the catalyst has about 1% to about 10% by weight of metal present on the support.

The catalyst support can be any solid, inert substance including, but not limited to, metal oxides such as silica, alumina, and titania, and carbons. The catalyst support can be in the form of powder, granules, pellets, or the like. The metal catalyst can also be a homogenous hydrogenation catalyst that dissolves in a solution or the substance to be hydrogenated. The homogeneous catalyst may consist of a combination of ligands and metal ions; in the case of charged species; counter ions may also be present.

Isolation of the intermediate compounds or products of the present inventive processes may be accomplished by techniques common to the art. The isolation techniques include but are not limited to filtration, distillation (including vacuum distillation and steam distillation), melt crystallization, solvent extraction, and sublimation. When filtration is used the desired product or intermediate may be the filtrate or the solid. One can optimize the precipitation of the products or by-products with the solvent composition. Vacuum distillation is the preferred method of distillation, as it decreases the amount of by-products.

The present invention further relates to methods to improve, enhance, or modify the flavor or fragrance of a product formulation comprising adding an effective amount of the compositions of the present invention to the product formulation.

The compositions of the present invention may be useful in both fine and functional perfumery. Articles where the compositions are of use as a perfuming ingredient include but are not limited to perfumes and colognes, soaps, shower and bath gels, shampoos and other hair-care products, body or air deodorants, detergents or fabric softeners or other household products.

Further, the compositions of the present invention may be useful for the flavor industry. The compositions can be used to flavor various articles including, but not limited to, foodstuffs, beverages, chewing gums, toothpaste or pharmaceutical preparations.

The present invention further relates to methods for modifying the rheology of an oil, hydrocarbon, petroleum or petroleum product, comprising adding an effective amount of the lactone compositions of the present invention to the oil, hydrocarbon, petroleum or petroleum product.

The present invention further relates to methods of formulating a cosmetic product comprising adding an effective amount of the lactone compositions of the present invention to the cosmetic product.

The present invention further relates to methods of formulating a liquid detergent or cleaning product comprising adding an effective amount of the lactone compositions of the present invention to the liquid detergent or cleaning product.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usage and conditions.

Common reagents were purchased from Sigma-Aldrich and solvents from VWR Scientific. Nuclear magnetic resonance (NMR) spectra were recorded on a Varian VXR-500 spectrometer. For reporting NMR data, the following abbreviations are used: s=singlet, d=doublet, t=triplet, q=quartet, m=multiplet, br=broad, dd=doublet of doublets, dt=doublet of triplets, etc.). Gas chromatography (GC) was performed on a Hewlett-Packard 6890 series instrument running HP Chemstation® software and equipped with a DB-5 capillary column from J&W Scientific (Length=10 m, Inner Diameter=0.1 mm, film thickness=0.17 micrometers). High-resolution mass spectral data were obtained on a Micromass Prospec magnetic sector GC mass spectrometer using methane chemical ionization and perfluorokerosene as an internal standard.

### Procedure 1

### Preparation of methyloxalyl-gamma-methyl-gamma-butyrolactone sodium salt

A 22-L flask equipped with a mechanical stirrer and nitrogen inlet was charged with diethyl oxalate (2409 g), gamma-methyl-gamma-butyrolactone (1419 g, Aldrich, St. Louis, Mo.), and methanol (3 L) and heated to 65° C. A 25 wt % solution of sodium methoxide in methanol (3.771 L) was added over 2 h. After complete addition, the slurry was held at 65° C. for one hour. After cooling to 25° C. and allowing to stand overnight, the slurry was filtered and the solid cake was washed with ethanol (Solids should precipitate on cooling slowly, but if not, seeding with approximately 50 g of product is necessary). The slurry was cooled to 10° C. The product was filtered, washed with approximately 2 L of cold methanol, and dried at 60° C. until it reached constant weight to give 2652 g (90% yield) of methyloxalyl-gamma-methyl-gamma-butyrolactone sodium salt as a white to pale yellow solid.

### Example 1

### Preparation of (E,Z)-3-ethylidene-5-methyl-dihydro-furan-2-one

A 1 L flask was charged with 25.0 g of methyloxalyl-gamma-methyl-gamma-butyrolactone sodium salt, 300 ml of denatured ethanol, and 12.6 g of distilled acetaldehyde. This mixture was stirred under a nitrogen atmosphere using a mechanical stirrer, and heated to 75° C. for 5 h. After cooling the mixture to room temperature, 200 mL of distilled water and 119 g of sodium bicarbonate were added and the mixture was stirred for 20 min. The product was extracted with methylene chloride (3×300 mL), and the extract was dried over anhydrous MgSO₄. After filtration through silica gel, the methylene chloride was removed from the filtrate using a rotary evaporator. The crude product was distilled under high vacuum (10⁻³-10⁻⁴ torr) and a fraction boiling at 81° C. yielded 6.1 g (40% yield) of (E,Z)-3-ethylidene-5-methyl-dihydro-furan-2-one as a colorless liquid. GC Purity=99%. ¹H NMR (500.9 MHz, CD₂Cl₂) (mixture of E and Z isomers with E/Z molar ratio=2.39): δ 6.69 (m, E isomer), 6.25 (m, Z isomer), 4.63 (m, E isomer), 4.56 (m, Z isomer), 2.99 (m), 2.66 (m), 2.46 (m, Z isomer), 2.40 (m, E isomer), 2.11 (dt, 7.4, 2.3 Hz, Z isomer), 1.83 (dt, 7.1, 2.0 Hz, E isomer), 1.38 (d, J=6.4 Hz, E isomer), 1.35 (d, J=6.2, Z isomer). ¹³C{¹H} NMR (126.0 MHz, CD₂Cl₂): δ 170.80, 170.08, 138.23, 135.29, 128.38, 126.32, 74.33, 74.11, 37.19, 33.07, 22.44, 21.95, 15.74, 14.04. High resolution GC mass spectral data: Theoretical (mass+H⁺) for C₇H₁₀O₂: 127.0759; Found: Two GC peaks observed owing to E and Z isomers: 127.0755, 127.0759.

### Example 2

### Preparation of (E,Z)-3-propylidene-5-methyl-dihydro-furan-2-one

(E,Z)-3-propylidene-5-methyl-dihydro-furan-2-one was prepared according to the procedure in Example 1 using 7.32 g of distilled propionaldehyde in place of acetaldehyde. After distillation under high vacuum (10⁻³-10⁻⁴ torr), 4.76 g (28% yield) of the colorless liquid product boiling at 89° C. was obtained. GC purity=97%. ¹H NMR (500.9 MHz, CD₂Cl₂) (mixture of E and Z isomers with E/Z ratio=1.34): δ 6.63 (tt, J=7.4, 3.0 Hz, E isomer), 6.15 (tt, J=7.9, 2.3 Hz, Z isomer), 4.62 (m, E isomer), 4.56 (m, Z isomer), 2.98 (m), 2.67 (quintuplet of t, J=7.5, 1.6 Hz, E isomer), 2.46 (m, Z isomer), 2.39 (m, E isomer), 2.18 (quintet of t, 7.4, 1.8 Hz), 1.48 (m), 1.38 (d, J=6.2 Hz, E isomer), 1.36 (d, J=6.2 Hz, E isomer), 1.07 (t, J=7.6 Hz, E isomer), 1.02 (t, J=7.6 Hz, Z isomer). ¹³C{¹H} NMR (126.0 MHz, CD2Cl2): δ 171.10, 169.95, 145.21, 141.85, 126.76, 125.04, 74.36, 74.18, 37.21, 33.10, 23.84, 22.43, 21.96, 21.39, 13.74, 12.83. Theoretical (mass+H⁺) for C₈H₁₂O₂: 141.0916; Found: Two GC peaks observed owing to E and Z isomers: 141.0910, 141.0911.

### Example 3

### Preparation of (E,Z)-3-butylidene-5-methyl-dihydro-furan-2-one

(E,Z)-3-butylidenle-5-methyl-dihydro-furan-2-one was prepared according to the procedure in Example 1 using 9.10 g of distilled 1-butyraldehyde in place of acetaldehyde. After distillation under high vacuum (10⁻³-10⁻⁴ torr), 9.09 g (49% yield) of the colorless liquid product boiling at 84° C. was obtained. GC purity=96%. ¹H NMR (500.9 MHz, CD₂Cl₂) (mixture of E and Z isomers with E/Z ratio=1.28): δ 6.65 (tt, J=7.7, 3.0 Hz, E isomer), 6.17 (tt, J=7.7, 2.2 Hz, Z isomer), 4.62 (m, E isomer), 4.56 (m, Z isomer), 2.99 (m), 2.64 (qt, J=7.5, 1.9 Hz, E isomer), 2.47 (m, Z isomer), 2.39 (m, E isomer), 2.15 (qt, 7.4, 1.8 Hz), 1.48 (m), 1.38 (d, J=6.3 Hz, E isomer), 1.36 (d, J=6.3 Hz, Z isomer), 0.94 (t overlapped with t, 7.5 Hz, E and Z isomers). ¹³C{¹H} NMR (125.8 MHz, CD₂Cl₂): δ 171.03, 170.01, 143.72, 140.42, 127.45, 125.66, 74.37, 74.14, 37.29, 33.28, 32.51, 29.84, 22.78, 22.42, 21.96 (2C), 14.00, 13.91. Theoretical (mass+H⁺) for C₉H₁₄O₂: 155.1072; Found: Two GC peaks observed owing to E and Z isomers: 155.1074, 155.1072.

### Example 4

### Preparation of (E,Z)-3-pentylidene-5-methyl-dihydro-furan-2-one

A 1 L flask was charged with 25.0 g methyloxalyl-gamma-methyl-gamma-butyrolactone sodium salt, 396 ml of denatured ethanol, and 10.8 g of distilled 1-valeroaldehyde. This mixture was stirred under a nitrogen atmosphere using a mechanical stirrer, and heated to 75° C. for 5 h. After cooling the mixture to room temperature, 500 ml of distilled water and 119 g of sodium bicarbonate were added and the mixture was stirred for 20 min. The product was extracted with methylene chloride (3×300 mL), and the extract was dried over anhydrous MgSO₄. After filtration through silica gel, the methylene chloride was removed from the filtrate using a rotary evaporator. The crude product was distilled under high vacuum (10⁻³-10⁻⁴ torr) yielding 5.95 g (29% yield) of 3-pentylidene-5-methyl-dihydro-furan-2-one as a colorless liquid. GC Purity=99%. ¹H NMR (500.9 MHz, CD₂Cl₂) (mixture of E and Z isomers with E/Z ratio=2.28): δ 6.64 (tt, J=7.6, 2.9 Hz, E isomer), 6.17 (tt, J=8.0, 2.2 Hz, Z isomer), 4.62 (m, E isomer), 4.56 (m, Z isomer), 2.99 (m), 2.66 (m), 2.46 (m, Z isomer), 2.39 (m, E isomer), 2.17 (qt, 7.4, 1.8 Hz), 1.49-1.32 (br m overlapped with d from E and Z isomers at δ 1.38 and 6 1.35, J=6.3 Hz), 0.92 (t overlapped with t at δ 0.91, 7.2 Hz, E and Z isomers). ¹³C{¹H} NMR (126.0 MHz, CD₂Cl₂): δ 170.97, 169.94, 143.90, 140.58, 127.29, 125.47, 74.32, 74.09, 37.26, 33.24, 31.67, 30.73, 30.19, 27.59, 22.79, 22.74, 22.42, 21.95, 14.07, 14.01. Theoretical (mass+H⁺) for C₁₀H₁₆O₂: 169.1229; Found: Two GC peaks observed owing to E and Z isomers: 169.1227, 169.1222.

### Example 5

### Preparation of (E,Z)-3-hexylidene-5-methyl-dihydro-furan-2-one

A 1 L flask was charged with 25.0 g methyloxalyl-gamma-methyl-gamma-butyrolactone sodium salt, 396 ml of denatured ethanol, and 12.6 g of distilled 1-hexanal. This mixture was stirred under a nitrogen atmosphere using a mechanical stirrer, and heated to 75° C. for 5 h. After cooling the mixture to room temperature, 500 ml of distilled water and 119 g of potassium bicarbonate were added and the mixture was stirred for 20 min. The product was extracted with methylene chloride (3×300 mL), and the extract was dried over anhydrous MgSO₄. After filtration through silica gel, the methylene chloride was removed on a rotary evaporator. The crude product was distilled under high vacuum (10⁻³-10⁻⁴ torr). A fraction boiling at 85° C. was collected yielding 13.8 g (63% yield) of 3-hexylidene-5-methyl-dihydro-furan-2-one as a mixture of E and Z stereoisomers. GC purity=98%. ¹H NMR (500.07 MHz, CD₂Cl₂) (mixture of E and Z isomers with E/Z ratio=1.25): E isomer: δ 6.650 (tt, J=7.6 and 3.1 Hz), 4.624(m), 2.984 (m), 2.388 (m), 2.160 (m), 1.478 (m), 1.382 (d, J=6.1 Hz), 1.314 (br m), 1.310 (br m), 0.893 (t, J=6.9 Hz); Z isomer: δ 6.166 (tt, J=7.6 and 2.1 Hz), 4.556 (m), 2.977 (m), 2.652 (m), 2.464 (m), 1.418, 1.352 (d, J=6.4 Hz), 1.310, 1.308, 0.886 (t, J=6.9 Hz). ¹³C{¹H} NMR (125 MHz, CD₂Cl₂) E isomer δ14.15, 22.41, 22.86, 28.27, 30.47, 31.88, 33.22, 74.37, 127.20, 140.75, 171.09; Z isomer δ 14.18, 21.95, 22.90, 27.84, 29.19, 31.85, 37.23, 74.14, 125.38, 144.05, 170.05. Theoretical (mass+H⁺) for C₁₁H₁₈O₂: 183.1385; Found: Two GC peaks observed owing to E and Z isomers: 183.1384, 183.1386.

TABLE 2
¹H and ¹³C{¹H} NMR spectroscopy resonance assignments of E
and Z isomers were made by analysis of Heteronuclear Single Quantum
Coherence and Heteronuclear Multiple Bond Correlation NMR spectra.
Resonances are in ppm downfield from tetramethylsilane internal
standard.

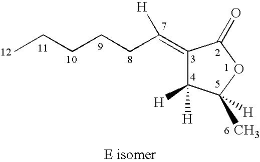

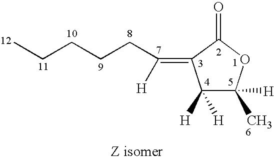

| | |
|---|---|
| E Isomer | Z Isomer |

| | Resonance | Resonance | Resonance | Resonance |
|---|---|---|---|---|
| Nucleus | in C₆D₆ | in CD₂Cl₂ | in C₆D₆ | in CD₂Cl₂ |
| H4a | 2.214 | 2.984 | 2.244 | 2.977 |
| H4b | 1.715 | 2.388 | 1.807 | 2.464 |
| H5 | 4.002 | 4.624 | 3.954 | 4.556 |
| H6 | 0.915 | 1.382 | 0.885 | 1.352 |
| H7 | 6.728 | 6.650 | 5.683 | 6.166 |
| H8 | 1.168 | 1.478 | 2.796 | 2.652 |
| H9 | 1.753 | 2.160 | 1.325 | 1.418 |
| H10 | 1.086 | 1.310 | 1.245 | 1.308 |
| H11 | 1.176 | 1.314 | 1.254 | 1.310 |
| H12 | 0.837 | 0.893 | 0.860 | 0.886 |
| C2 | 169.916 | 171.094 | 169.009 | 170.048 |
| C3 | 127.491 | 127.204 | 125.669 | 125.384 |
| C4 | 32.594 | 33.220 | 36.654 | 37.234 |
| C5 | 73.016 | 74.369 | 72.882 | 74.137 |
| C6 | 22.014 | 22.413 | 21.569 | 21.951 |
| C7 | 139.286 | 140.749 | 142.702 | 144.053 |
| C8 | 28.059 | 28.267 | 27.652 | 27.840 |
| C9 | 30.076 | 30.467 | 29.196 | 29.186 |
| C10 | 31.672 | 31.883 | 31.719 | 31.848 |
| C11 | 22.755 | 22.864 | 22.839 | 22.901 |
| C12 | 14.128 | 14.150 | 14.206 | 14.182 |

### Example 6

### Preparation of (E,Z)-3-Heptylidene-5-methyl-dihydro-furan-2-one

(E,Z)-3-Heptylidene-5-methyl-dihydro-furan-2-one was prepared according to the procedure in Example 5 using 14.4 g of distilled 1-heptanal in place of 1-hexanal. After distillation under high vacuum, 3.52 g (15% yield) of the colorless liquid product was obtained. GC purity=99%. ¹H NMR (499.9 MHz, CD₂Cl₂) (mixture of E and Z isomers with E/Z ratio=2.80): δ 6.65 (tt, J=7.7, 2.9 Hz, E isomer), 6.17 (tt, J=7.8, 2.3 Hz, Z isomer), 4.62 (m, E isomer), 4.55 (m, Z isomer), 3.00 (m), 2.97 (m), 2.66 (ddt, J=15.1, 7.4, 1.9 Hz), 2.46 (m, Z isomer), 2.39 (m, E isomer), 2.16 (qt, 7.6, 1.6 Hz), 1.51-1.27 (br m overlapped with d from E and Z isomers at 6 1.38 and δ 1.35, J=6.3 Hz), 0.89 (t overlapped with t, 6.9 Hz, E and Z isomers). ¹³C{¹H} NMR (125.7 MHz, CD₂Cl₂): δ 170.99, 169.92, 143.95, 140.66, 127.25, 125.44, 74.32, 74.09, 37.28, 33.27, 32.06, 32.04, 30.50, 29.50, 29.40, 29.33, 28.59, 27.92, 22.96 (2C), 22.41, 21.96, 14.23 (2C). Theoretical (mass+H⁺) for C₁₂H₂₀O₂: 197.1542; Found: Two GC peaks observed owing to E and Z isomers: 197.1538, 197.1544.

### Example 7

### Preparation of (E,Z)-3-octylidene-5-methyl-dihydro-furan-2-one

(E,Z)-3-octylidene-5-methyl-dihydro-furan-2-one was prepared according to the procedure in Example 1 using 19.4 g of methyloxalyl-gamma-methyl-gamma-butyrolactone sodium salt, 305 ml of denatured ethanol, and 12.0 g of distilled 1-octyl aldehyde in place of acetaldehyde. After distillation under high vacuum, a fraction boiling at 110° C. yielded 9.75 g (50% yield) of the colorless liquid. GC purity=99%. ¹H NMR (500.3 MHz, CD₂Cl₂) (mixture of E and Z isomers with E/Z ratio=1.88): δ 6.64 (tt, J=7.6, 2.8 Hz, E isomer), 6.16 (tt, 0.29 H, J=7.7, 2.3 Hz, Z isomer), 4.62 (m, E isomer), 4.55 (m, Z isomer), 2.98 (m), 2.66 (m, Z isomer), 2.46 (m, Z isomer), 2.39 (m, E isomer), 2.16 (qt, 7.4, 1.8 Hz, E isomer), 1.51-1.25 (br m overlapped with d from E and Z isomers at δ 1.38 and δ 1.35, J=6.3 Hz), 0.88 (t overlapped with t, 7.1 Hz, E and Z isomers). ¹³C{¹H} NMR (125.8 MHz, CD₂Cl₂): δ 170.95, 169.91, 143.95, 140.63, 127.19, 125.38, 74.27, 74.05, 37.24, 33.22, 32.19, 32.15, 30.47(2), 29.66, 29.61, 29.50, 29.45, 28.58, 27.87, 23.00 (2C), 22.38, 21.92 (2C). Theoretical (mass+H⁺) for C₁₃H₂₂O₂: 211.1698; Found: Two GC peaks observed owing to E and Z isomers: 211.1700, 211.1693.

### Example 8

### Preparation of (E,Z)-3-nonylidene-5-methyl-dihydro-furan-2-one

(E,Z)-3-nonylidene-5-methyl-dihydro-furan-2-one was prepared according the procedure in Example 4 using 17.0 g of 1-nonyl aldehyde in place of 1-valeroaldehyde. After distillation under high vacuum, a fraction boiling at 115° C. yielded 14.5 g (54% yield) of the colorless liquid. GC purity=98%. ¹H NMR (499.9 MHz, CD₂Cl₂) (mixture of E and Z isomers with E/Z ratio=1.69): δ 6.65 (m, E isomer), 6.16 (m, Z isomer), 4.62 (m, E isomer), 4.55 (m, Z isomer), 2.98 (m), 2.66 (m, Z isomer), 2.46 (m, Z isomer), 2.39 (m, E isomer), 2.16 (qt, 7.4, 1.7 Hz, E isomer), 1.59-1.24 (br m overlapped with d from E and Z isomers at δ 1.38 and δ 1.35, J=6.2 Hz), 0.88 (br triplet, J=7.1 Hz, E and Z isomers). ¹³C{¹H} NMR (125.7 MHz, CD₂Cl₂): δ 171.0, 170.0, 144.0, 140.7, 127.2, 125.4, 74.3, 74.1, 44.3, 37.3, 33.3, 32.3, 30.5, 29.8, 29.8, 29.7, 29.7, 29.7, 29.6, 29.5, 28.6, 27.9, 23.1, 22.5, 22.4, 21.9, 14.2 (2C). Theoretical (mass+H⁺) for C₁₄H₂₄O₂: 225.1855; Found: Two GC peaks observed owing to E and Z isomers: 225.1859, 225.1845.

### Example 9

### Preparation of (E,Z)-3-decylidene-5-methyl-dihydro-furan-2-one

(E,Z)-3-decylidene-5-methyl-dihydro-furan-2-one was prepared according to the procedure in Example 1 using 12.5 g of ethyloxalyl-gamma-methyl-gamma-butyrolactone sodium salt, 200 ml of denatured ethanol, and 9.35 g of distilled 1-decyl aldehyde in place of 1-acetaldehyde. After distillation under high vacuum, a fraction boiling at 118° C. yielded 8.74 g (61% yield) of the colorless liquid. GC purity=98%. ¹H NMR (499.9 MHz, CD₂Cl₂) (mixture of E and Z isomers with E/Z ratio=1.53): δ 6.65 (m, E isomer), 6.16 (m, Z isomer), 4.62 (m, E isomer), 4.55 (m, Z isomer), 2.98 (m), 2.66 (m, Z isomer), 2.46 (m, Z isomer), 2.39 (m, E isomer), 2.16 (qt, 7.3, 1.6 Hz, E isomer), 1.59-1.23 (br m overlapped with d from E and Z isomers at δ 1.38 and δ 1.35, J=6.3 Hz), 0.88 (br triplet, J=7.1 Hz, E and Z isomers). ¹³C{¹H} NMR (125.7 MHz, CD₂Cl₂): δ 171.03, 169.98, 144.05, 140.74, 127.22, 125.41, 74.33, 74.11, 37.30, 33.29, 32.34, 32.31, 30.53, 29.98, 29.94, 29.90, 29.84, 29.75 (2C), 29.71 (2C), 29.57, 28.62, 27.94, 23.10 (2C), 22.43, 21.97, 14.28 (2C). Theoretical (mass+H⁺) for C₁₅H₂₆O₂: 239.201 1; Found: Two GC peaks observed owing to E and Z isomers: 239.2022, 239.2003.

### Example 10

### Preparation of (E,Z)-3-(3,5,5-trimethylhexylidene)-5-methyl-dihydrofuran-2-one

(E,Z)-3-(3,5,5-trimethylhexylidene)-5-methyl-dihydrofuran-2-one was prepared according the procedure in Example 4 using 17.0 g of 3,3,5-trimethyl-1-hexanal in place of 1-valeroaldehyde. After distillation under high vacuum, a fraction boiling at 113° C. yielded 11.5 g (43% yield) of the colorless liquid. GC purity=97%. ¹H NMR (500.9 MHz, CD₂Cl₂) (mixture of E and Z isomers with E/Z ratio=1.68): δ 6.67 (tt, J=7.7, 2.9 Hz, E isomer), 6.18 (m, Z isomer), 4.62 (m, E isomer), 4.56 (m, Z isomer), 2.99 (m), 2.60 (m), 2.48 (m, Z isomer), 2.38 (m, E isomer), 2.17 (m), 2.03 (m), 1.76 (m, E isomer), 1.66 (m, Z isomer), 1.38 (dd, J=6.3, 0.8 Hz, E isomer), 1.36 (dd, J=6.3, 1.4 Hz, Z isomer), 1.27 (m), 1.11 (m), 0.96 (br m), 0.90 (br m). ¹³C{¹H} NMR (126.0 MHz, CD₂Cl₂): δ 170.85 (2C), 169.95 (2C), 142.95 (2C), 139.61 (2C), 128.02 (2C), 126.19, 126.18, 74.27 (2C), 74.03, 74.01, 51.07, 51.04, 50.89, 50.87, 40.07 (2C), 37.37, 37.35, 37.00, 36.98, 33.50 (2C), 31.33 (2C), 30.13 (8C), 30.11(8C), 29.77 (2C), 22.77, 22.75, 22.71, 22.68, 22.42 (2C), 21.97(2C). Theoretical (mass+H⁺) for C₁₄H₂₄O₂: 225.1855; Found: (two GC peaks observed owing to E and Z isomers) 225.1851, 225.1850.

### Example 11

### Preparation of cis-3-ethyl-5-methyl-dihydro-furan-2-one

In a nitrogen-filled glove box, a 100 mL round bottomed flask equipped with a Teflon®-coated magnetic stirring bar was charged with 2.0 g of (E,Z)-3-ethylidene-5-methyl-dihydro-furan-2-one (prepared as in Example 1), 0.210 g of 10% Palladium on Carbon catalyst (Aldrich Chemical Company), 40.0 mL of denatured ethanol, and 10 ml of methanol. This was placed on a high vacuum line and degassed. It was then stirred overnight under one atmosphere of dihydrogen gas. he catalyst was then removed by filtration and the solvent was removed on a rotary evaporator Analysis of the crude product by GC indicated a >99% conversion and >97% yield to the desired product. The crude product was placed in an oil sublimer and was sublimed on a high vacuum line (10⁻³-10⁻⁴ torr) using a heated oil bath to yield 1.12 g of product (55% yield; some loss of crude product was observed owing to incomplete transfer of liquid from vessel to vessel and losses owing to holdup on the condenser and glass wall of the oil sublimer). GC purity=97%. ¹H NMR (500.9 MHz, CD₂Cl₂): δ 4.46 (m, 1 H), 2.54 (m, 1 H), 2.45 (m, 1 H), 1.87 (m, 1 H), 1.52-1.37 (m overlapped with d at δ 1.38, 5 H), 0.97 (t, 3 H, J=7.6 Hz). ¹³C{¹H} NMR (126.0 MHz, CD₂Cl₂): δ 178.98, 75.38, 43.25, 36.76, 23.78, 21.17, 11.81. Theoretical (mass+H⁺) for C₇H₁₂O₂: 129.0916; Found: 129.0915.

### Example 12

### Preparation of cis-3-propyl-5-methyl-dihydro-furan-2-one

The compound cis-3-propyl-5-methyl-dihydro-furan-2-one was prepared by the procedure of Example 11 using 2.0 g of (E,Z)-3-propylidene-5-methyl-dihydro-furan-2-one (prepared by the procedure of Example 2) yielding crude product that by GC analysis indicated a >99% conversion and >97% yield to the desired product. After oil sublimation, 1.05 g of a colorless liquid was obtained (52% yield; some loss of crude product was observed owing to incomplete transfer of liquid from vessel to vessel and losses owing to holdup on the condenser and glass wall of the oil sublimer). GC purity=99%. ¹H NMR (500.9 MHz, CD₂Cl₂): δ 4.44 (m, 1 H), 2.58 (m, 1 H), 2.46 (m, 1 H), 1.83 (m, 1 H), 1.48-1.33 (m overlapped with d at δ1.38 J=6.0 Hz, 7 H), 0.94 (t, 3 H, J=7.4 Hz). ¹³C{¹H} NMR (126.0 MHz, CD₂Cl₂): δ 179.21, 75.43, 41.65, 37.40, 32.93, 21.17, 21.00, 14.03. Theoretical (mass+H⁺) for C₈H₁₄O₂: 143.1072; Found: 143.1065.

### Example 13

### Preparation of cis-3-butyl-5-methyl-dihydro-furan-2-one

The compound cis-3-butyl-5-methyl-dihydro-furan-2-one was prepared by the procedure of Example 11 using 2.0 g of (E,Z)-3-butylidene-5-methyl-dihydro-furan-2-one (prepared by the procedure of Example 3) yielding crude product that by GC analysis indicated a >99% conversion and >97% yield to the desired product. After oil sublimation, 1.04 g of a colorless liquid was obtained (51% yield; some loss of crude product was observed owing to incomplete transfer of liquid from vessel to vessel and losses owing to holdup on the condenser and glass wall of the oil sublimer). GC purity=95%. ¹H NMR (500.9 MHz, CD₂Cl₂): δ 4.44 (m, 1 H), 2.56 (m, 1 H), 2.46 (m, 1 H), 1.86 (m, 1 H), 1.48-1.31 (m overlapped with d at δ 1.38 J=6.1 Hz, 9 H), 0.91 (t, 3 H, J=7.3 Hz). ¹³C{¹H} NMR (126.0 MHz, CD₂Cl₂): δ 179.21, 75.43, 41.87, 37.42, 30.49, 29.98, 22.93, 21.18, 14.11. Theoretical (mass+H⁺) for C₉H₁₆O₂: 157.1229; Found: 157.1225.

### Example 14

### Preparation of cis-3-pentyl-5-methyl-dihydro-furan-2-one

The compound cis-3-pentyl-5-methyl-dihydro-furan-2-one was prepared by the procedure of Example 11 using 2.0 g of (E,Z)-3-pentylidene-5-methyl-dihydro-furan-2-one (prepared by the procedure of Example 4) yielding crude product that by GC analysis indicated a >99% conversion and >97% yield to the desired product. After oil sublimation, 1.49 g of a colorless liquid was obtained (74% yield; some loss of crude product was observed owing to incomplete transfer of liquid from vessel to vessel and losses owing to holdup on the condenser and glass wall of the oil sublimer). GC purity=95%. ¹H NMR (500.9 MHz, CD₂Cl₂): δ 4.44 (m, 1 H), 2.57 (m, 1 H), 2.46 (m, 1 H), 1.86 (m, 1 H), 1.48-1.31 (m overlapped with d at δ1.38, 11 H), 0.90 (t, 3 H, J=6.7 Hz). ¹³C{¹H} NMR (126.0 MHz, CD₂Cl₂): δ 179.19, 75.41, 41.87, 37.39, 31.99, 30.73, 27.44, 22.89, 21.15, 14.16. Theoretical (mass+H⁺) for C₁₀H₁₈O₂: 171.1385; Found: 171.1384.

### Example 15

### Preparation of cis-3-hexyl-5-methyl-dihydro-furan-2-one

In a nitrogen-filled glove box, a 100 mL flask was charged with 2.00 g of 3-hexylidene-5-methyl-dihydro-furan-2-one (mixture of E and Z stereoisomers), 0.210 g of 10% palladium on carbon catalyst (Aldrich Chemical Company), 40 mL of denatured ethanol, and 10 mL of methanol. The flask was attached to a high vacuum line via an adapter, and the apparatus was degassed. An atmosphere of dihydrogen gas was admitted to the flask, and gas uptake was monitored using a mercury manometer connected to the vacuum line. After 12 hours, the hydrogen gas and solvents were removed in vacuo. The solution containing the product was filtered from the catalyst, and solvents were removed in vacuo yielding crude product that by GC analysis indicated a >99% conversion and >97% yield to the desired product. The crude product was purified using an oil sublimation apparatus under high vacuum with heating. The product was obtained as a liquid which solidifies upon standing (melting point=28.5° C.). GC purity=97%. Yield: 1.50 g (74% yield; some loss of crude product was observed owing to incomplete transfer of liquid from vessel to vessel and losses owing to holdup on the condenser and glass wall of the oil sublimer). ¹H NMR (500.07 MHz, CD₂Cl₂): δ 4.40 (m, 1 H), 2.57 (m, 1 H), 2.46 (m 1 H), 1.85 (br m, 1 H), 1.45 (m, 1 H), 1.385 (d, J=6.1 Hz, 3 H), 1.40-1.25 (br m, 9 H), 0.89 (t, J=7.1 Hz, 3 H). ¹³C{¹H} NMR (125.7 MHz, CD₂Cl₂): δ 179.50, 75.73, 42.20, 37.76, 32.43, 31.15, 29.83, 28.09, 23.35, 21.51, 14.57. Theoretical (mass+H⁺) for C₁₁H₂₀O₂: 185.1542; Found: 185.1546.

TABLE 3
¹H and ¹³C chemical shift assignments for cis-3-hexyl-5-methyl-
dihydro-furan-2-one in C₆D₆ were obtained by analysis of Heteronuclear
Single Quantum Coherence and Heteronuclear Multiple Bond Correlation
NMR spectra.

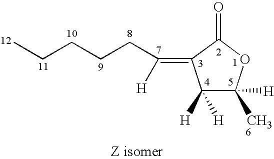

| ¹H Labels | ¹H Numbers | ¹H Shifts | ¹³C Numbers | ¹³C Shifts |
|---|---|---|---|---|
| B | H3 | 2.024 | C2 | 177.307 |
| D1 | H4a | 1.615 | C3 | 41.103 |
| D2 | H4b | 0.852 | C4 | 36.638 |
| A | H5 | 3.788 | C5 | 73.744 |
| C | H6 | 0.977 | C6 | 20.511 |
| E1 | H7a | 1.835 | C7 | 30.376 |
| E2 | H7b | 1.186 | C8 | 27.304 |
| F | H8 | 1.150 | C9 | 29.112 |
| G | H9 | 1.161 | C10 | 31.705 |
| H | H10 | 1.186 | C11 | 22.670 |
| I | H11 | 1.258 | C12 | 13.979 |
| J | H12 | 0.898 | | |

The cis stereochemistry for the methyl and hexyl groups on the lactone ring for the compound prepared in this example was established by NMR Nuclear Overhauser Effects spectroscopy studying cross relaxation rates and signal enhancement owing to magnetization transfer upon selective inversion of single resonances observed by NMR spectroscopy. This allowed calculation of distances between proton sites and comparison to distances expected from ab initio molecular calculations for both the cis and trans isomers. The data shown in Table 3 established that the methyl and hexyl groups were in a cis configuration on the lactone ring for this composition

TABLE 4
Comparison of selected inter-proton distances in
cis- and trans-3-hexyl-5-methyl-dihydro-furan-2-one
with distances derived from cross-relaxation rates
from NOE NMR experiments. Units are in Angstroms
(10⁻⁸ cm). The NOE distances are based on a calculated
D1-D2 distance of 1.79 Å.

| | d(A-B) | d(A-C) | d(A-D1) | d(A-D2) |
|---|---|---|---|---|
| Ab initio trans | 3.94 | 2.71 | 2.82 | 2.46 |
| Ab initio cis | 2.87 | 2.71 | 2.44 | 3.08 |
| NOE | 2.64 | 2.67 | 2.30 | 2.83 |

### Example 16

### Preparation of cis-3-heptyl-5-methyl-dihydro-furan-2-one

The compound cis-3-heptyl-5-methyl-dihydro-furan-2-one was prepared by the procedure of Example 11 using 1.9 g of (E,Z)-3-heptylidene-5-methyl-dihydro-furan-2-one (prepared by the procedure of Example 6). The crude product was isolated by sublimation yielding crude product that by GC analysis indicated a >99% conversion and >97% yield to the desired product. After sublimation, 0.399 g of a colorless solid was obtained (21% yield; some loss of crude product was observed owing to incomplete transfer of material from vessel to vessel). GC purity=99.7%. ¹H NMR (499.9 MHz, CD₂Cl₂): δ 4.44 (m, 1 H), 2.57 (m, 1 H), 2.45 (m, 1 H), 1.86 (m, 1 H), 1.48-1.29 (m overlapped with d at δ 1.38, 15 H), 0.89 (t, 3 H, J=6.8 Hz). ¹³C{¹H} NMR (125.7 MHz, CD₂Cl₂): δ 179.20, 75.41, 41.90, 37.43, 32.24, 30.82, 29.79, 29.55, 27.80, 23.06, 21.18, 14.26. Theoretical (mass+H⁺) for C₁₂H₂₂O₂: 199.1698; Found: 199.1691.

### Example 17

### Preparation of cis-3-octyl-5-methyl-dihydro-furan-2-one

The compound cis-3-octyl-5-methyl-dihydro-furan-2-one was prepared by the procedure of Example 11 using 2.0 g of (E,Z)-3-octylidene-5-methyl-dihydro-furan-2-one (prepared by the procedure of Example 7). The crude product was obtained with 99% conversion and in 97% yield. The product was isolated by sublimation yielding 1.55 g of a colorless solid (77% yield; some loss of crude product was observed owing to incomplete transfer of material from vessel to vessel). GC purity=99%. ¹H NMR (500.3 MHz, CD₂Cl₂): δ 4.43 (m, 1 H), 2.57 (m, 1 H), 2.45 (m, 1 H), 1.85 (m, 1 H), 1.48-1.28 (m overlapped with d at δ 1.38, 17 H), 0.88 (t, 3 H, J=7.0 Hz). ¹³C{¹H} NMR (125.8 MHz, CD₂Cl₂): δ 179.16, 75.37, 41.85, 37.37, 32.25, 30.77, 29.79 (2 C), 29.63, 27.76, 23.05, 21.13, 14.24. Theoretical (mass+H⁺) for C₁₃H₂₄O₂: 213.1855; Found: 213.1858.

### X-Ray Crystal Structure Analysis of cis-3-octyl-5-methyl-dihydro-furan-2-one

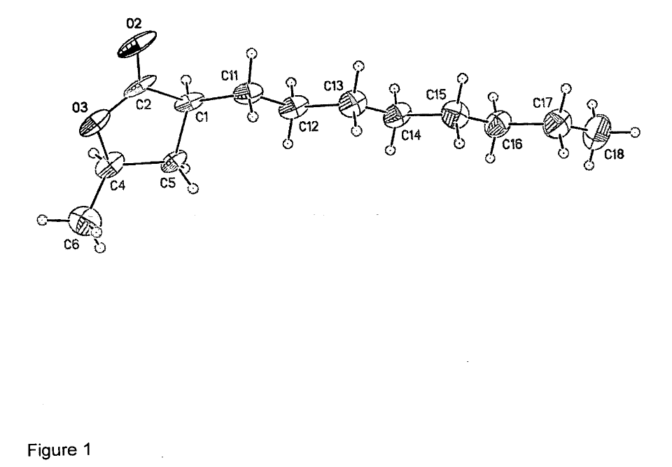

CRYSTAL DATA: C₁₃H₂₄O₂, from sublimation, colorless, irregular plate, ˜0.150×0.150×0.020 mm, monoclinic, P21/c, a=28.29(2) Å, b=4.708(4) Å, c=9.812(8) Å, beta=98.948(19)°, Vol=1290.9(18) Å³, Z=4, T=−100° C., Formula weight=212.32, Density=1.092g/cm³, μ(Mo)=0.07mm⁻¹.
DATA COLLECTION: Bruker SMART 1K CCD system, MoKalpha radiation, standard focus tube, anode power=45 kV×40 mA, crystal to plate distance=4.9 mm, 512×512 pixels/frame, hemisphere data acquisition, total scans=4, total frames=1330, oscillation/frame=−0.30°, exposure/frame=30.0 sec/frame, maximum detector swing angle=−28.0°, beam center=(255.25,253.13), in plane spot width=0.00, omega half width=0.00, SAINT integration, hkl min/max=(−37, 32, −6, 6, −12, 11), data input to shelx=7790, unique data=3041, two-theta range=4.38 to 56.44°, completeness to two-theta 56.44=95.30%, R(int-xl)=0.1685, SADABS correction applied.
SOLUTION AND REFINEMENT: Structure was solved using XS(Shelxtl), refined using shelxtl software package, refinement by full-matrix least squares on F², scattering factors from International. Tables, Vol C Tables 4.2.6.8 and 6.1.1.4, number of data=3041, number of restraints=0, number of parameters=139, data/parameter ratio=21.88, goodness-of-fit on F²=1.06, R indices[I>4sigma(I)]R1=0.1107, wR2=0.2483, R indices(all data) R1=0.3035, wR2=0.3184, max difference peak and hole=0.410 and −0.323 e/Å³, All of the hydrogen atoms have been idealized using a riding model. The rotation of the methyl groups are refined. ORTEP diagram is shown in FIG. 1.

### FIG. 1

FIG. 1 describes the structure of cis-3-octyl-5-methyl-dihydro-furan-2-one as determined by X-ray crystallography analysis. Structure demonstrates the cis orientation of the octyl and methyl groups on the lactone ring. The asymmetric unit contains one molecule as shown with thermal ellipsoids drawn to the 50% probability level.

### Example 18

### Preparation of cis-3-(3,5,5-trimethylhexyl)-5-methyl-dihydro-furan-2-one

The compound cis-3-(3,5,5-trimethylhexyl)-5-methyl-dihydro-furan-2-one was prepared by the procedure of Example 11 using 2.0 g of (E,Z)-3-(3,5,5-trimethylhexylidene)-5-methyl-dihydrofuran-2-one (prepared by the procedure of Example 10) yielding crude product that by GC analysis indicated a >99% conversion and >97% yield to the desired product. After oil sublimation,1.12 g of a colorless liquid was obtained (53% yield; some loss of crude product was observed owing to incomplete transfer of liquid from vessel to vessel and losses owing to holdup on the condenser and glass wall of the oil sublimer). GC purity=97%. ¹H NMR (499.9 MHz, CD₂Cl₂): δ 4.43 (m, 1 H), 2.54 (m, 1 H), 2.45 (m, 1 H), 1.88 (m, 1 H), 1.56-1.02 (m overlapped with d at δ1.38, 11H), 0.94-0.87 (m overlapped with overlapping d at δ 0.94 and δ 0.93, 12 H, J=5.5 Hz). ¹³C{1H} NMR (125.7 MHz, CD₂Cl₂): δ 179.11, 179.06, 75.39, 75.37, 51.58, 51.36, 42.16, 42.01, 37.53, 37.45 (3C), 31.31 (2C), 30.16 (6C), 29.72, 29.51, 28.48 (2C), 22.80, 22.56, 21.16 (2C). Theoretical (mass+H⁺) for C₁₄H₂₆O₂: 227.201 1; Found: 227.2018.

### Example 19

### Preparation of (E,Z)-3-cyclohexylmethylidene-5-methyl-dihydrofuran-2-one

A flask was charged with 14.5 g of oxalyl methyloxalyl-gamma-methyl-gamma-butyrolactone sodium salt, 175 ml of ethanol, and 7.82 g of cyclohexylcarboxaldehyde (Aldrich Chemical Co., distilled). This was stirred with a mechanical stirrer, under nitrogen, at 75° C. for 36 h. It was then allowed to cool to room temperature. To this was added 69.0 g of sodium bicarbonate and 100.0 ml of water. After stirring for 20 min, it was then extracted with methylene chloride (3×200 mL) and dried over magnesium sulfate. It was then filtered through silica gel and solvent was removed on a rotary evaporator to yield 9.6 g of crude product. The crude product was distilled under high vacuum (10⁻³-10⁻⁴ torr) and a fraction boiling at 108° C. yielded 1.27 g of (E,Z)-3-cyclohexylmethylidene-5-methyl-dihydrofuran-2-one as a colorless liquid (9% yield). GC Purity=97%. ¹H NMR (500 MHz, CD₂Cl₂) (mixture of E and Z isomers with E/Z ratio=1.0): δ 6.50 (tt, J=9.8, 2.8 Hz, E isomer), 5.97 (tt, J=9.8, 2.3 Hz, Z isomer), 4.61 (m), 4.55 (m), 3.38 (m), 2.98 (m), 2.43 (m), 2.18 (m), 1.79-1.62 (br m), 1.42-1.02 (br m, overlapped with two d at δ1.38, J=6.6 Hz and at δ1.35, J=6.0 Hz. ¹³C{¹H} NMR (126 MHz, CD₂Cl₂): δ171.40, 169.78, 148.95, 145.25, 125.43, 123.75, 74.33, 74.11, 39.79, 37.27, 36.21, 33.09, 32.95, 32.82, 31.95, 31.86, 26.33, 26.21, 25.91 (4C), 22.39, 21.94. High resolution GC mass spectral data: Theoretical (mass+H⁺) for C₁₂H₁₈O₂: 195.1385; Found: 195.1367.

### Example 20

### Preparation of cis-3-cyclohexylmethyl-5-methyl-dihydro-furan-2-one

In a nitrogen-filled glove box, a 100 mL round bottomed flask equipped with a Teflon®-coated magnetic stirring bar was charged with 1.041 g of (E₁Z)-3-cyclohexylmethylidene-5-methyl-dihydro-furan-2-one (prepared as in Example 19), 0.105 g of 10% palladium on carbon catalyst (Aldrich Chemical Company), 20.0 mL of denatured ethanol, and 5 ml of methanol. This was placed on a high vacuum line and degassed. It was then stirred overnight under one atmosphere of dihydrogen gas. The catalyst was then removed by filtration and the solvent was removed on a rotary evaporator. Analysis of the crude product indicated >99% conversion and 97% yield. The crude product was placed in a sublimer and was sublimed on a high vacuum line (10⁻³-10⁻⁴ torr) using a heated oil bath to yield 0.509 g of product as a white solid (47% yield; some loss of crude product was observed owing to incomplete transfer of material from vessel to vessel). Melting point=60-62° C. GC purity=98%. ¹H NMR (500.9 MHz, CD₂Cl₂): δ 4.43 (m, 1 H), 2.65 (m, 1 H), 2.47 (m, 1 H), 1.79-1.58 (m, 6 H), 1.46-1.12 (m overlapped with d at δ1.38 J=6.5 Hz, 9 H), 0.99 (m, 1 H), 0.89 (m, 1 H). ¹³C{¹H} NMR (126.0 MHz, CD₂Cl₂): δ 171.40, 169.78, 148.95, 145.25, 125.43, 123.75, 74.33, 74.11, 39.79, 37.27, 36.21, 33.09, 32.95, 32.82, 31.95, 31.86, 26.33, 26.21, 25.91 (4C), 22.39, 21.94. Theoretical (mass+H⁺) for C₁₂H₂₀O₂: 197.1542; Found: 197.1545.

### Example 21

### Preparation of (5S)-cis-3-hexylidene-5-methyl-dihydro-furan-2-one

The compound (5S)-5-methyl-dihydro-furan-2-one is prepared by the procedure reported in the literature: Hedenstroem, Erik; Hoegberg, Hans-Erik; Wassgren, Ann-Britt; Bergstroem, Gunnar; Loefqvist, Jan; Tetrahedron; 48; 1992; pp. 3139-3146. The (5S)-3-methyloxalyl-5-methyl-dihydro-2-furanone sodium salt is prepared from this compound according to Procedure 1 using (5S)-5-methyl-dihydro-furan-2-one in place of gamma-methyl-gamma-butyrolactone. The (5S)-cis-3-hexylidene-5-methyl-dihydro-furan-2-one is prepared from this salt according to the procedure of Example 5 using this salt in place of methyloxalyl-gamma-methyl-gamma-butyrolactone sodium salt.

### Example 22

### Preparation of (3R,5S)-cis-3-hexyl-5-methyl-dihydro-furan-2-one

The compound (3R,5S)-cis-3-hexyl-5-methyl-dihydro-furan-2-one is prepared from (5S)-cis-3-hexylidene-5-methyl-dihydro-furan-2-one (Example 21) by hydrogenation according to the procedure of Example 15.

### Example 23

### Preparation of (5R)-cis-3-hexylidene-5-methyl-dihydro-furan-2-one

The compound (5R)-5-methyl-dihydro-furan-2-one is prepared by the procedure reported in the literature using (R)-propylene oxide: Hedenstroem, Erik; Hoegberg, Hans-Erik; Wassgren, Ann-Britt; Bergstroem, Gunnar; Loefqvist, Jan; Tetrahedron; 48; 1992; pp. 3139-3146. The (5R)-3-methyloxalyl-5methyl-dihydro-2-furanone sodium salt is prepared from this compound according to Procedure 1 using (5R)-5-methyl-dihydro-furan-2-one in place of gamma-methyl-gamma-butyrolactone. The (5R)-cis-3-hexylidene-5-methyl-dihydro-furan-2-one is prepared from this salt according to the procedure of Example 5 using this salt in place of methyloxalyl-gamma-methyl-gamma-butyrolactone sodium salt.

### Example 24

### Preparation of (3S,5R-cis-3-hexyl-5-methyl-dihydro-furan-2-one

The compound (3S,5R)-cis-3-hexyl-5-methyl-dihydro-furan-2-one is prepared from (5R)-cis-3-hexylidene-5-methyl-dihydro-furan-2-one (Example 23) by hydrogenation according to the procedure of Example 15.

What is claimed is:

1. A process for preparing a compound represented by formula I: 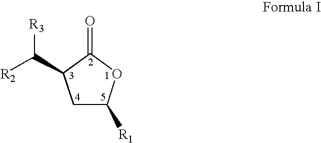
wherein, the group at position 3 of the lactone ring (containing R₂ and R₃), and R₁ have a cis orientation with respect to each other; R₁ comprises a group selected from the groups consisting of linear, branched, cyclic, bicyclic, saturated and unsaturated hydrocarbyl groups; R₂, and R₃ are independently selected from the groups consisting of hydrogen and linear, branched, cyclic, bicyclic, saturated and unsaturated hydrocarbyl groups; and said composition contains a molar ratio of cis:trans stereoisomers greater than 49:1; said process comprising the steps:
(a) contacting a lactone of formula II with an oxalic acid diester in the presence of a base and a solvent to form an intermediate mixture comprising a compound of formula III and isolating the compound of formula III from the intermediate mixture;
(b) treating the isolated compound of formula III with an aldehyde or ketone, to form a second intermediate mixture comprising a compound of formula IV and isolating the compound of formula IV from the second intermediate mixture; and
(c) hydrogenating the compound of formula IV in the presence of a catalyst and optionally a solvent to form a product mixture comprising a compound of formula I and isolating a pure compound of formula I from the product mixture; 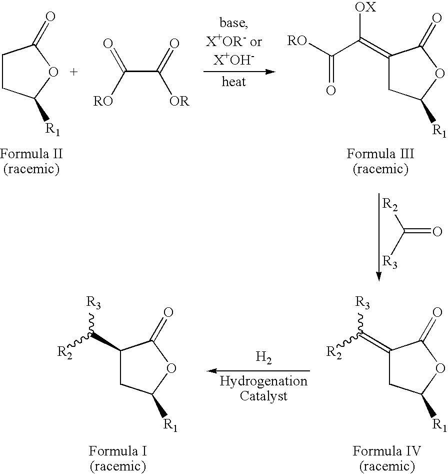
wherein, R is a hydrocarbyl or substituted hydrocarbyl group, and X⁺ is a cation.

2. The process of claim 1 wherein the purity of the isolated compound of formula I is at least about 95 percent by gas chromatographic analysis.

3. A composition of matter comprising a compound of formula l: 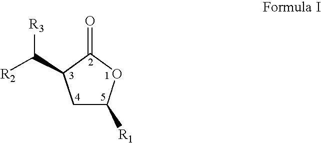
wherein, the group at position 3 of the lactone ring (containing R₂ and R₃), and R₁ have a cis orientation with respect to each other; R₁ comprises a group selected from the groups consisting of linear, branched, cyclic, bicyclic, saturated and unsaturated hydrocarbyl groups; R₂, and R₃ are independently selected from the groups consisting of hydrogen and linear, branched, cyclic, bicyclic, saturated and unsaturated hydrocarbyl groups excluding the compound wherein R₂ and R₃ are both H, when R₁ is methyl or phenyl; said composition contains a molar ratio of cis:trans stereoisomers greater than 49:1; and said composition is greater than 95 percent enantiomerically pure cis, being the (3S,5S), (3R,5R), (3S,5R), or (3R,5S) optically pure isomer.

4. The composition of matter of claim 3, wherein the overall purity by gas chromatographic analysis is at least about 95 percent.

5. A process for preparing the enantiomerically pure composition of claim 3 or 4 comprising the steps:
(a) contacting an optically pure stereoisomer of a lactone of formula II with an oxalic acid diester in the presence of a base and a solvent to form an intermediate mixture comprising an optically pure compound of formula III and isolating the optically pure compound of formula III from the intermediate mixture;
(b) treating the isolated optically pure compound of formula III with an aldehyde or ketone, to form a second intermediate mixture and isolating an optically pure compound of formula IV from the second intermediate mixture; and
(c) hydrogenating the optically pure compound of formula IV in the presence of a catalyst and optionally a solvent to form a product mixture and isolating an enantiomerically pure compound of formula I from the product mixture; 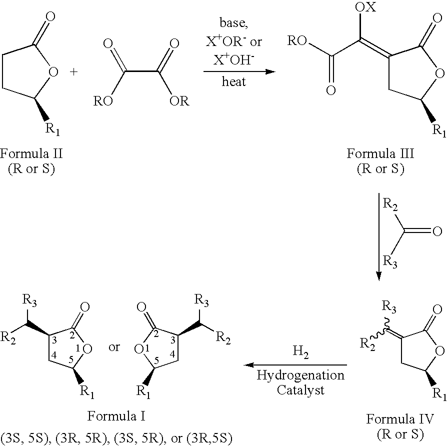
wherein, R is a hydrocarbyl or substituted hydrocarbyl group, and X⁺ is a cation.

6. A method to improve, enhance, or modify the flavor or fragrance of a product formulation comprising adding an effective amount of the composition of claim 3 or 4 to said product formulation.

7. A method to improve or modify the rheology of an oil, hydrocarbon, petroleum or petroleum product comprising adding an effective amount of the composition of claim 3 or 4 to said oil, hydrocarbon, petroleum or petroleum product.

8. A method of formulating a cosmetic product comprising adding an effective amount of the composition of claim 3 or 4 to said cosmetic product.

9. A method of formulating a liquid detergent or cleaning product comprising adding an effective amount of the composition of claim 3 or 4 to said liquid detergent or cleaning product.
